(19) United States
(12) Patent Application Publication
(10) Pub. No.: US 2007/0016130 A1
(43) Pub. Date: Jan. 18, 2007

(54) Complex Shaped Steerable Catheters and Methods for Making and Using Them

(76) Inventors: Stephen A. Leeflang, Sunnyvale, CA (US); George A. Morrison, Austin, TX (US); Nicholas J. Mourlas, Mountain View, CA (US); Christian S. Eversull, Palo Alto, CA (US)

Correspondence Address: Vista IP Law Group LLP, 2040 MAIN STREET, 9TH FLOOR, IRVINE, CA, 92614, US

(21) Appl. No.: 11/382,026

(22) Filed: May 6, 2006

Related U.S. Application Data
(60) Provisional application No. 60/678,517, filed on May 6, 2005.
(60) Provisional application No. 60/752,763, filed on Dec. 20, 2005.

(51) Int. Cl. A61M 31/00; A61B 1/06

(52) U.S. Cl. 604/95.04; 600/160

(57) ABSTRACT

Apparatus and methods are provided for accessing a body lumen within a patient's body. Generally, the apparatus includes a tubular member including proximal and distal ends, a steering element extending between the proximal and distal ends, and an actuator for directing the distal end between a relaxed configuration and a complex curved configuration. In the relaxed configuration, the distal end may assume a straight or curved shape. In the complex curved configuration, the distal end may assume a curvilinear shape. In one embodiment, the complex curved configuration may include a first curved portion defining an arc within a plane, and a second portion that extends out of the plane. An embodiment with a left hand rule configuration may be introduced into the right atrium of a heart from a superior approach, and the complex curved configuration may facilitate accessing the coronary sinus.

This application claims benefit of provisional application Ser. Nos. 60/678,517, filed May 6, 2005 and Ser. No. 60/752,763, filed Dec. 20, 2005. The entire disclosures of these applications are expressly incorporated herein by reference.

### FIELD OF THE INVENTION

The present invention relates generally to catheters for introduction into body lumens within a patient's body, and, more particularly, to complexly shaped catheters for accessing body lumens, cavities, and/or visualization within a patient's body and to methods for constructing and using such catheters.

### BACKGROUND

Minimally invasive procedures have been implemented in a variety of medical settings, e.g., for vascular interventions, such as angioplasty, stenting, embolic protection, electrical heart stimulation, heart mapping and visualization, tissue ablation, and the like. One such procedure involves delivering an electrical lead into a coronary vein of a patient's heart that may be used to electrically stimulate the heart. Another procedure involves delivering an electrode probe into a patient's heart to ablate tissue, e.g., surrounding the pulmonary ostia to treat atrial fibrillation.

In accordance with one embodiment, an apparatus is provided for treating a condition within the patient's heart. A patient's heart anatomy has been shown to vary, especially when the patient suffers from various heart related afflictions, e.g., chronic heart failure. The geometry of the venous system leading to and including the right atrium may vary widely between patients, as may the origin and/or trajectory of the coronary sinus. Taken together, these variations make transvenous coronary sinus access challenging, e.g., to deliver a catheter, lead, or other device into the coronary sinus. Patients undergoing such procedures may suffer hardship given available devices, e.g., because of the delay or other difficulty in accessing the coronary sinus. In some situations, because such access may be monitored tactilely or using two-dimensional imaging, such as fluoroscopy, a physician may be unable to access the coronary sinus in some anatomy. Thus, patients and physicians would benefit from an apparatus that can accommodate complex anatomical variations.

Accordingly, apparatus and methods for delivering devices into a patient's vasculature, e.g., the coronary sinus, or other body lumens would be useful.

### SUMMARY OF THE INVENTION

The present invention is directed generally to apparatus and methods for accessing body lumens within a patient's body and/or visualization within a patient's body and to methods for making and using such apparatus. More particularly, the present invention is directed to complexly shaped catheters for accessing body lumens and/or cavities, apparatus including such catheters, and methods for making and using them.

In accordance with one embodiment, an apparatus is provided that includes a flexible tubular member including a proximal end and distal end sized for introduction into a body lumen, and a steerable distal portion. The distal portion maybe controlled using one or more actuators, e.g., on a handle on the proximal end. In one embodiment, the proximal end of the tubular member may be substantially rigid or otherwise may remain substantially straight relative to the distal end. The distal portion may be deflectable through a simple arc, e.g., using a pull-wire or other mechanism. In addition or alternatively, the distal portion may be shaped, i.e., the material of the distal portion may have a shape set into the material, thereby biasing the distal portion to assume a predetermined shape, twist, and/or other deflection when free from external forces.

In one embodiment, the distal portion may be biased towards a curvilinear shape and/or may include a torsion or twist. A desired combination of predetermined shape and/or deflection characteristics, as well as a steering mechanism, may be selected to facilitate positioning the distal end of the tubular member relative to an anatomical feature within a body cavity.

In an exemplary embodiment, the predetermined shape of the distal portion may include a bend within a first plane, e.g., biased to an angle of approximately thirty degrees (30°) relative to a substantially straight portion of the tubular member proximal to the bend. In addition, the distal portion may be steerable, e.g., such that the distal end may be directed out of the first plane. The distal portion may be steerable within a second plane intersecting the first plane, e.g., at an angle up to approximately ninety degrees (90°). Alternatively, the bend and/or the deflection of the distal portion may be more complex than defining a single plane.

In one embodiment, the bend may be defined by the left hand rule for helixes. For example, the direction of rotation of the distal portion may be represented by fingers of the left hand curving outward from the hand with the thumb defining, when extended from the hand, the general direction of propagation. Such a configuration may be particularly useful for cannulating the coronary sinus from a superior approach, i.e., when the right atrium is accessed from the superior vena cava. Specifically, a left handed helical deflection creates a direction vector at the tip of the device that matches the generally anatomically posterior entry vector of the coronary sinus ostium. Matching the tip vector with the entry vector facilitates much improved cannulation as compared to a simple tip curvature that may succeed in placing the tip of the device at the coronary sinus ostium but does not approach the sinus at a sufficiently ideal entry direction to facilitate cannulation. A left handed helical deflection is especially necessary when the location of the ostium relative to the entry plane of the device through superior vena cava exacerbates the mismatch of the entry vector of the sinus with the approach direction of a simple deflectable or shape set catheter.

In accordance with another embodiment, any of the apparatus described herein may include a transparent expandable member, e.g., a balloon or other membrane, on the distal end, and/or an imaging assembly including a distal lens, e.g., disposed proximal to or otherwise within the transparent expandable member. A bend, twist, or other deflection may be programmed into the distal portion proximal to the expandable member and/or the imaging system. The section of the tubular member extending beyond the bend may be generally straight and/or substantially rigid, e.g., which may maintain a view angle of an imaging lens of the imaging system substantially aligned with a distal face of the expandable member.

In addition or alternatively, the distal portion of the apparatus may be steerable or otherwise deflected, which may be limited to the distal portion proximal to the bend and/or proximal to the expandable member and/or imaging system.

In accordance with yet another embodiment, an apparatus is provided that includes a flexible distal portion including multiple predetermined bends set therein and may be steerable in one or more planes. A distal end of the apparatus, e.g., beyond the bends and/or steerable portions, may include a transparent expandable member, imaging assembly, and/or other components. For example, the distal portion may include a first bend having an angle of approximately thirty degrees (30°) relative to a substantially straight portion of the tubular member proximal to the first bend, and may steerable such that the distal portion may be directed up to approximately ninety (90°) degrees out of the plane formed by the straight proximal tubular member and the distal tubular member when deflected. In addition, the distal portion may include a second bend defining a twist, e.g., that is counter clock-wise to form a corkscrew or helical area in the distal portion of the tubular member. In addition, the distal portion may include a third portion, e.g., defining a posterior curve within another plane.

The corkscrew or other twisted portion of the distal portion may vary in length and/or location on the distal portion, depending on intended anatomical structures of a body cavity into which the apparatus is to be delivered. For example, if the apparatus approaches the right atrium of a heart from a superior position, the distal portion may be configured to move in a slight rightward inferior motion as it approaches the coronary sinus and then curve left in a posterior motion of deflection. This configuration may allow the tubular member to be manipulated through anatomical structures of a body cavity, e.g., through the right atrium into the coronary sinus.

The apparatus may be formed from one or more materials that may have one or more bends programmed therein, may be deflected or otherwise steered, while remaining flexible and structurally intact.

More particularly, the first bend may be defined by the left hand rule. The direction of rotation of the distal portion may be defined by fingers of the left hand curving outward from the hand with the thumb defining the general direction of propagation when extended from the hand. The catheter may also be deflectable further as described elsewhere herein, the combination of deflection and multiple bends providing a range of complex shapes of the distal portion.

In a further embodiment, the deflectable distal portion may be deflected to form an approximately helical configuration. For example, when cannulating the coronary sinus from a superior approach, this helical configuration may follow the left hand rule where the thumb points distally along a longitudinal axis of the apparatus, and the curled fingers representing the bend may wrap in the direction of the helical curve adopted by the distal portion of the apparatus. Alternatively, it may be desirable to configure the distal portion according to a right hand rule, e.g., if accessing the coronary sinus from an inferior approach, i.e., via the inferior vena cava.

In yet an alternative embodiment of the twisted and/or helical shape, the apparatus may be constructed such that the distal portion includes a twist, e.g., linear in pitch, such that deflection of a distal portion of the apparatus in at least one plane is substantially linear throughout most of the deflection of the distal portion. Alternatively, the steerable distal portion of the apparatus may deflect orthogonally to the direction of deflection in at least one plane such that the tip position defines a substantially linear pathway throughout most of the deflection. This configuration may allow the distal tip to be maintained within an open space of an atrium as it transitions from a straight undeflected configuration towards a desired complex curved configuration, e.g., for cannulating the coronary sinus or other body lumen, as described elsewhere herein.

In addition, this configuration may allow the apparatus to reach a desired shape without substantial interference with the boundaries of the atrium or body cavity. Second, the substantially linear tip deflection in at least one plane, may promote ease of understanding the position of the tip given the complex curve of the apparatus, e.g., while monitoring the apparatus using a two-dimensional imaging or reference system, such as fluoroscopy or other x-ray system.

In a further embodiment, the apparatus may be deflectable to substantially form two arcs defining approximately perpendicular planes. Optionally, if desired, the arcs may follow the left hand rule, the first arc curving in the direction of the curved thumb, and the second arc curving in the direction of the curved fingers of the left hand.

In yet another alternative, any of the apparatus described herein may also include a slidable inner member disposed adjacent to a pull wire and/or otherwise adapted to modulate a location at which the pull wire may cause the distal portion to begin deflection, for example, as described elsewhere herein. In combination with the helical and/or double arc configurations, the pullwire or an inner member may be used to induce approximately lateral motion of the distal tip of the apparatus, e.g., approximately perpendicular to the motion caused by deflection. The radius of curvature, spiral diameter, and/or other dimensions of the distal portion may be adjusted using the slidable inner adjustment member.

In a further embodiment, the helical or double arc configuration may be combined with one or more bends along the length of the catheter to generate further complex geometry.

Any embodiment of the apparatus described herein may also include a transparent expandable member, e.g., at its distal tip, and/or an imaging system having a distal lens disposed proximally within the expandable member.

In accordance with another embodiment, an apparatus is provided for treating a condition within a patient's heart that includes a flexible tubular member including a proximal end, a distal end sized for introduction in to a body lumen, and a substantially transparent expandable member carried by the distal end of the tubular member. The proximal end of the tubular member may remain substantially straight and/or rigid relative to the distal end. The proximal end may merge with a complex curved distal end, which may be shaped to seat the tubular member relative to an anatomical feature within a body cavity.

The complexly shaped distal end may have a first portion that may be curved out of plane relative to the proximal end. The out of plane curve may be anterior and left, demonstrating a curvature with a gradual deflection of up to forty degrees (40°) from the plane of the proximal end. The gradual curvature deflection may have an appearance of an S-shape, an L-shape, a J-shape, and/or some combination in which the shape provides an alternative angle that is out of alignment with the direct plane of the proximal end. The tubular member may be formed from any substantially flexible and/or semi-rigid material, which may be deflectable while remaining flexible and structurally intact.

In accordance with still another embodiment, apparatus and/or method are provided for cannulating a coronary sinus within a patient's heart from a superior approach. The apparatus may include a flexible tubular member including a proximal end, and a distal end sized for introduction into a body lumen. The distal end may be steerable and also preformed such that, through the combination of steering and the pre-shape, a range of complex shapes may be selectively achievable upon actuation from the proximal end. The distal end of the tubular member body may be steerable when attached to a steerable catheter handle. Exemplary steerable catheter handles may be found in co-pending application Ser. No. 11/062,074, filed Feb. 17, 2005, the entire disclosure of which is expressly incorporated herein by reference.

Optionally, the distal end may include a substantially transparent expandable member carried by the distal end of the tubular member, and an optical imaging assembly carried by the distal end of the tubular member and at least partially surrounded by the expandable member for imaging tissue structures beyond the distal end through the expandable member. A preformed distal end of the expandable member may facilitate the optical imaging assembly keeping the image in alignment with the distal end of the tubular member. The optical imaging assembly and apparatus and methods for making and using them may be found in co-pending application Ser. No. 11/062,074, filed Feb. 17, 2005, the disclosure of which is expressly incorporated by reference herein.

In accordance with still another embodiment, a method is provided for making a complex curved distal portion for a catheter or other apparatus. An elongate tubular body may be provided sized for introduction into a patient's body, and a shape may be set into the body such that the body remains flexible but may be biased to the shape. A pullwire or other steering mechanism may be directed through the body and fixed adjacent one end. The body may include an extrusion or other core, and an outer layer surrounding the core.

In one embodiment, the body may be twisted about its central axis, and the twist set into the body, while in another embodiment, the body may be set into a simple curved shape, a helical shape, and the like. The body may include a passage extending along the body but offset radially from a longitudinal axis of the body, and the steering mechanism may be inserted through the passage and fixed at one end of the body. When the steering mechanism is subjected to axial force, e.g., pulling or pushing the steering mechanism from an end opposite the fixed end, the steering mechanism may cause the body to assume a complex curved configuration, such as those described elsewhere herein.

In addition to including the catheters described herein as an apparatus for accessing body lumens, cavities, and/or recesses, the catheters may also be used for other medical procedures within a patient's body. For example, the catheters may be included in a delivery system, wherein the complex curved distal end may be modified to carry a needle with stem cells, medicaments, and the like. Alternatively, the apparatus may carry an energy probe or other instrument disposed on or deployable through the tubular member. For example, the probe may be used for delivering electrical, laser, thermal, or other energy to tissue in the region beyond the tissue structure. In yet a further alternative, the apparatus may include one or more electrodes for recording electrical signals.

Other aspects and features of the present invention will become apparent from consideration of the following description taken in conjunction with the accompanying drawings.

### BRIEF DESCRIPTION OF THE DRAWINGS

The drawings illustrate exemplary embodiments of the invention, in which:

FIG. 1 is a side view of an apparatus, including an imaging catheter having a handle on a proximal end, a balloon on a distal end, a syringe for expanding the balloon, and a monitor for displaying images obtained by the catheter through the balloon.

FIG. 2 is a side view of the catheter of the apparatus of FIG. 1.

FIG. 3 is a side view detail of the distal end of the catheter of FIG. 1, with the balloon in an expanded condition.

FIGS. 4A-4E are cross-sectional views of the catheter of FIG. 2 taken along lines 4A-4A, 4B-4B, 4C-4C, 4D-4D, and 4E-4E, respectively.

FIG. 5 is a cross-sectional view of an alternative embodiment of a core that may be provided within a catheter, such as those described herein.

FIGS. 6-8 are perspective views from different angles of a first embodiment of a distal end of a catheter, showing the distal end assuming a complex curved shape by actuating a steering mechanism of the catheter.

FIG. 9 is a side view of the first embodiment of FIGS. 6-8 with the distal end in a relaxed, substantially straighten configuration.

FIG. 10 is another side view of the first embodiment of FIGS. 6-8, showing the distal end in a second complex curved shape achieved by adjusting a steering adjustment member within the distal end.

FIG. 11 is a side view of a second embodiment of a distal end of a catheter with the distal end in a relaxed, substantially straight configuration.

FIGS. 12-14 are side views of the second embodiment of FIG. 11, showing various configurations of the distal end achieved by actuating a steering mechanism of the catheter.

FIGS. 15 and 16 are top and side views, respectively, of a third embodiment of a distal end of a catheter with the distal end in a relaxed configuration including a slight curve.

FIGS. 17 and 18 are top and side views, respectively, of the third embodiment of FIGS. 15 and 16 with the distal end directed to a complex curved configuration.

FIG. 19 is a top view of the third embodiment, with the distal end directed to another complex curved configuration achieved by adjusting a steering adjustment member within the distal end.

FIG. 20 is a cross-sectional view of a heart, showing a catheter being introduced into the right atrium.

FIG. 21 is a side view of a set of lines representing pathways defined by modeled trajectories through several patients' hearts into the coronary sinus.

FIG. 22 is a top view of the pathways shown in FIG. 21, i.e., in a plane orthogonal to FIG. 21.

FIG. 23 is a detail of the pathways shown in FIG. 21.

FIG. 24 includes a series of top views of a distal end of a catheter, showing the distal end being directed from a substantially straight configuration to a complex curved configuration.

FIG. 25A is a simplified anterior view of a heart, showing the superior vena cava, right atrium, and coronary sinus.

FIG. 25B is a cross-sectional view of the heart of FIG. 25A taken along line 25B-25B, showing axes of the coronary sinus relative to the superior vena cava.

### DETAILED DESCRIPTION

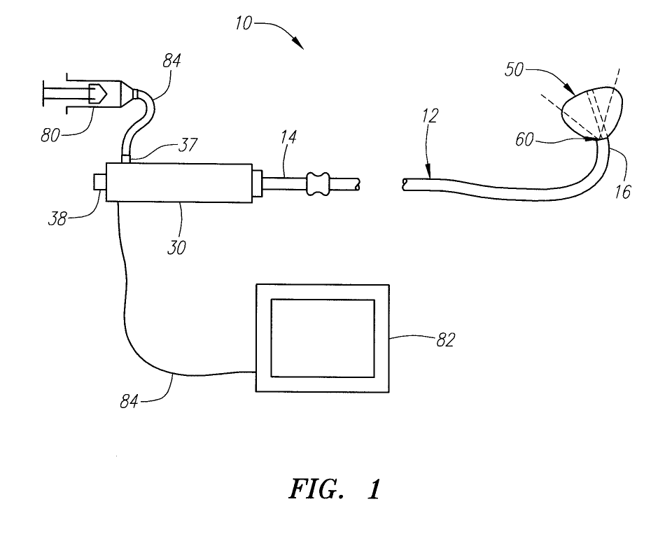

Turning to the drawings, FIGS. 1-3 show a first embodiment of an apparatus 10 for imaging a body lumen, e.g., for visualizing, cannulating, and/or accessing a body lumen from a body cavity (not shown). As explained further below, the apparatus 10 may be used for imaging a wall of a body lumen, e.g., a right atrium of a heart, e.g., for visualizing, accessing, and/or cannulating a coronary sinus ostium. Alternatively, the apparatus 10 may be used for visualizing, accessing, and/or cannulating other body lumens, e.g., for delivering one or more therapeutic and/or diagnostic agents into tissue, and/or for puncturing through tissue to access a region beyond the punctured tissue. As used herein, “body lumen” may refer to any passage within a patient's body, e.g., an artery, vein, or other blood vessel, or a body cavity, such as a chamber within a patient's heart, e.g., a ventricle or atrium. Although exemplary embodiments are described herein, additional information that may relate to the structure and/or methods for making and/or using the apparatus 10 may also be found in co-pending applications Ser. Nos. 10/447,526, filed May 29, 2003, Ser. No. 11/057,074, filed Feb. 11, 2005, and Ser. No. 11/062,074, filed Feb. 17, 2005. The entire disclosures of these references are expressly incorporated by reference herein.

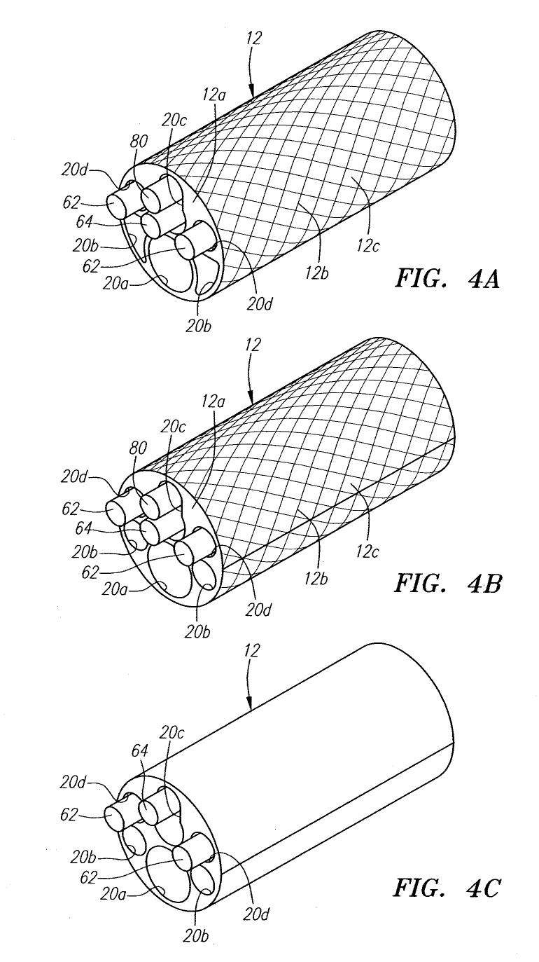

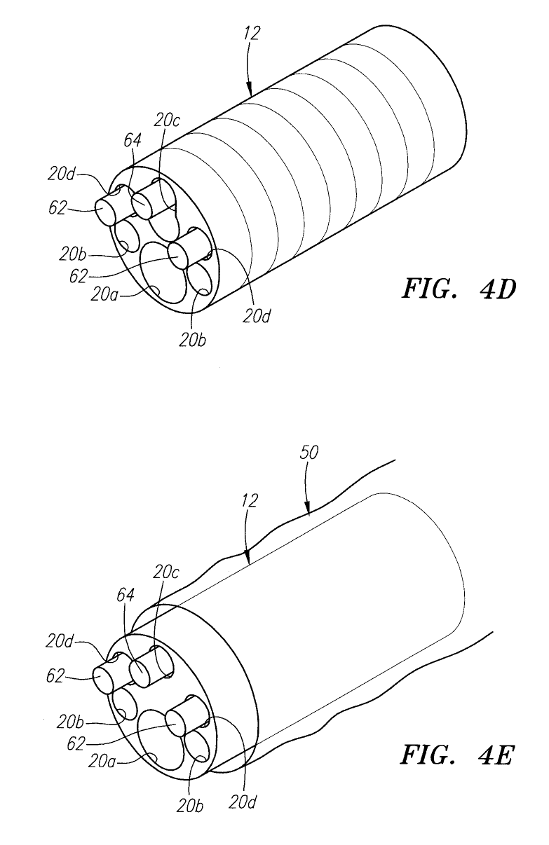

Generally, as shown in FIG. 1, the apparatus 10 includes a catheter or other elongate member 12, including a handle 30 on a proximal end 14 of the catheter 12, and a balloon or other expandable member 50 on a distal end 16 of the catheter 12. An imaging assembly 60 may be provided on or otherwise carried by the catheter 12 for imaging through the balloon 50, e.g. including one or more illumination fibers 62 and/or imaging optical fibers 64 (not shown in FIG. 1, see, e.g., FIGS. 4A-4E) extending through the catheter 12, as described further below. Optionally, the apparatus 10 may include other components, e.g., a syringe or other source of inflation media 80, a monitor or other output device 82, and the like. In additional embodiments, the apparatus 10 may include other devices that may be delivered through, over (e.g., a sheath over the catheter 12), or otherwise advanced from the catheter 12, e.g., a guidewire, a needle, a guide catheter, a lead, an energy probe, and the like (not shown).

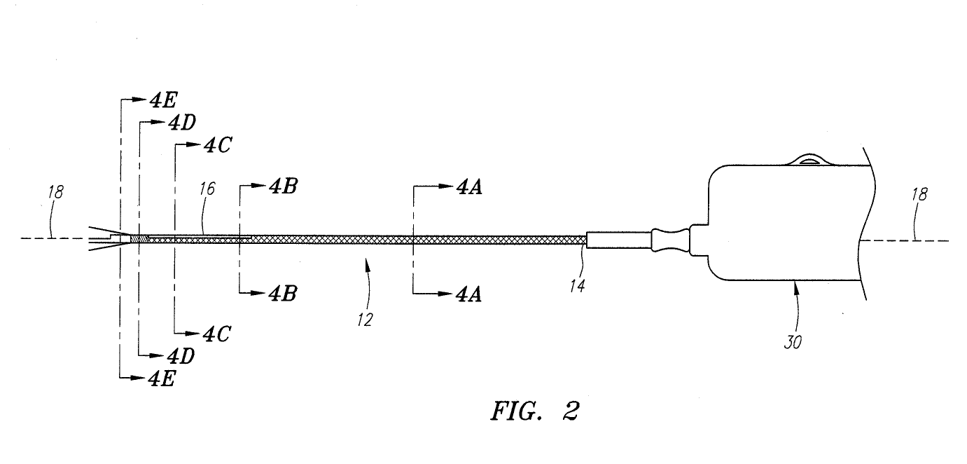

Turning to FIG. 2, the catheter 12 generally includes an elongate tubular body including a proximal end 14, a distal end 16 sized and/or shaped for insertion into a patient's body, and a central longitudinal axis 18 extending between the proximal and distal ends 14, 16. As best seen in FIGS. 4A-4E, the catheter 12 may include one or more lumens 20 extending between the proximal and distal ends 14, 16. For example, the catheter 12 may include an accessory lumen 20a, one or more inflation lumens 20b (two shown), and one or more lumens 20c, 20d for components of the imaging assembly 60, e.g., one or more light fibers 62 (two shown) and/or imaging fibers 64 (one shown). Optionally, the catheter 12 may include one or more additional lumens (not shown) extending at least partially between the proximal and distal ends 14, 16, e.g., for one or more separate steering elements (not shown). In exemplary embodiments, the catheter 12 may have a diameter between about four and ten French (1.33-3.33 mm), or between about six and eight French (2.00-2.67 mm). In alternative embodiments, the catheter 12 may be used with a guide wire, e.g., having a diameter of not more than about 0.035 inch (0.88 mm) or less.

The catheter 12 may be substantially flexible, semi-rigid, and/or rigid along its length, and may be formed from a variety of materials, including plastic, metal, and/or composite materials. For example, the catheter 12 may be substantially flexible at the distal end 16, e.g., to facilitate steering and/or advancement through tortuous anatomy, and/or may be semi-rigid or substantially rigid at the proximal end 14, e.g., to enhance pushability of the catheter 12 without substantial risk of buckling or kinking. In addition, as described further below, the distal end 16 may include a bend, twist, deflection, or other shape programmed or set into the distal end 16. Thus, the distal end 16 may assume the predetermined shape in a relaxed condition, e.g., free from external forces, but may be substantially flexible so that the distal end 16 may be directed from the relaxed condition, e.g., using a steering mechanism and/or when directed through tortuous anatomy.

In an exemplary embodiment, the catheter 12 may be formed from PEBAX, which may include a braid or other reinforcement structure therein. For example, as shown in FIGS. 4A and 4B, the catheter 12 may include a plastic core 12a, e.g., polyurethane, extruded or otherwise formed with the lumens 20 therein, over which a braid 12b, e.g., of metal, plastic, or composite fibers, may be disposed. A tube of PET 12c (partially cut away in FIG. 2B) may be disposed around the braid-covered core 12a, and then heat shrunk or otherwise attached to capture and/or secure the braid 12b between the tube 12c and the core 12a. Optionally, an adhesive may be used to bond one or more of the layers 12a-12c of the catheter 12 together.

Optionally, the plastic core 12a may include a composite construction. For example, a portion of the catheter 12 adjacent the distal end 16 may include semi-cylindrical portions of different materials that may be secured together to provide a tubular body. For example, the last several inches, e.g., up to eight inches, adjacent to the distal end 16 may include upper and lower halves or portions (not shown) that may be bonded or otherwise secured together or formed in a divided extrusion process. In an exemplary embodiment, the upper half, e.g., including imaging fibers 62, 64, may be made from polyurethane, and the lower half, including the accessory lumen 20a and/or inflation lumens 20b, may be made from PEBAX. Alternatively, the upper half may be made from a lower durometer PEBAX and the lower half from a higher durometer PEBAX. Alternatively, other materials may be selected having differential physical properties to facilitate directionality or biasing of deflection of the catheter 12, as described elsewhere herein. For example, such composite construction may provide a desired off-axis center of modulus or hinge, as explained further below.

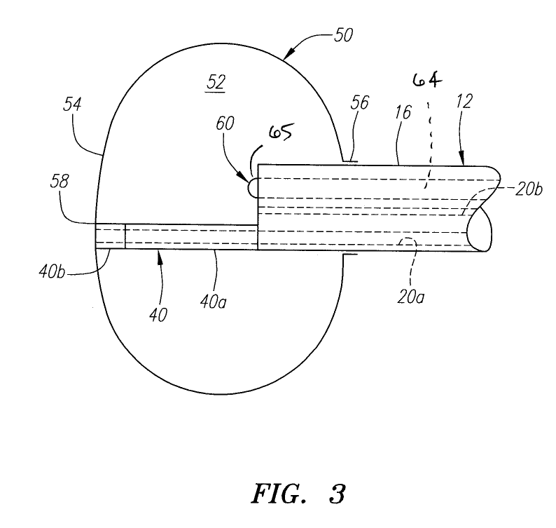

Optionally, as shown in FIG. 3, the catheter 12 may include a tubular extension 40 that extends distally from the distal end 16. The tubular extension 40 has a diameter or other cross-section that is substantially smaller than the catheter 12. In addition, the tubular extension 40 may be offset from or concentric with the central axis 18 of the catheter 12. The tubular extension 40 may facilitate balloon stabilization and/or may maximize a field of view of the imaging assembly 60, as explained elsewhere herein. The tubular extension 40 may include a section of hypotube, coil, or other tubular material, e.g., formed from metal, plastic, or composite materials. In an exemplary embodiment, the tubular extension 40 may include a first section 40a formed from a substantially rigid material, e.g., stainless steel, and a second tip section 40b formed from a flexible material, e.g., PEBAX, to provide a relatively soft and/or atraumatic tip for the apparatus 10. In a further exemplary embodiment, the tubular extension 40 may include a first section 40a formed from a semi-rigid material, e.g. coiled steel flat wire, and a second tip section 40b formed from a flexible material, e.g. silicone. Such a tip section 40b may reduce abrasion or other tissue damage while moving the tubular extension 40 along tissue during use, as explained further below.

The first section 40a may be at least partially inserted into the distal end 16 of the catheter 12, e.g., into the accessory lumen 20a. For example, the material of the distal end 16 may be softened to allow the material to reflow as the first section 40a of the tubular extension is inserted into the accessory lumen 20a. Alternatively, the distal end 16 may include a recess (not shown) sized for receiving a portion of the first section 40a therein. In addition or alternatively, the first section 40a may be attached to the distal end 16 by bonding with adhesive, using mating connectors and/or an interference fit, and the like. The second section 40b may be bonded or otherwise attached to the first section 40a before or after the first section 40a is attached to the distal end 16 of the catheter 12.

Turning to FIGS. 1 and 4A-4E, the imaging assembly 60 generally includes an objective lens 66 that is exposed within an interior 52 of the balloon 50 for capturing light images through the balloon 50. The objective lens 66 may be coupled to an optical imaging fiber 64, e.g. a coherent image bundle, that extends between the proximal and distal ends 14, 16 of the catheter 12, e.g., through the lumen 20d, as shown in FIGS. 4A-4E. The imaging fiber 64 may include a plurality of individual optical fibers, e.g., between about one thousand and one hundred fifty thousand (1,000-150,000) fibers, or between about three thousand and ten thousand (3,000-10,000) fibers, in order to provide a desired resolution in the images obtained by the optical fiber 64. The material of the imaging fiber 64 may be sufficiently flexible to bend as the catheter 12 bends or is otherwise deflected.

A device, e.g., a display 82, computer or other device (not shown) may be coupled or otherwise provided at the proximal end 14 of the apparatus 10 for acquiring, capturing, and/or displaying images transmitted by the imaging fiber 64. For example, as shown in FIG. 2, a lens 65 may be coupled to the fiber bundle 64 for focusing and/or resolving light passing through the imaging fiber 64, e.g., to pass the image to the display 82. The lens 65 may provide sufficient magnification to prevent substantial loss of resolution, which may depend upon the pixel density of the device 68. For example, a lens 65 having magnification between about 1.3× and 3× may spread a single pixel from the optical fiber 64 onto four or more pixels on the device 68, which may sufficiently reduce resolution loss.

The device coupled to the fiber bundle 64 may include a CCD, CMOS, and/or other device, e.g., to digitize or otherwise convert the light images from the imaging fiber 64 into electrical signals that may be transferred to a processor and/or display. The device may be coupled to the monitor 82, e.g., by a cable 84, as shown in FIG. 1. In addition or alternatively, the device may be used to store the images acquired from the imaging fiber 64. Additional information on capture devices that may be used may be found in application Ser. No. 10/447,526, incorporated by reference herein.

The imaging assembly 60 may also include one or more illumination fibers or light guides 62 carried by the distal end 16 of the catheter 12 for delivering light into the interior 52 and/or through a distal surface 54 of the balloon 50. As shown in FIGS. 4A-4E, a pair of illumination fibers 62 may be provided in the catheter 12. The illumination fibers 62 may be spaced apart from one another, e.g., in separate lumens 20d to minimize shadows, which may be cast by the tubular extension 40. A source of light (not shown) may be coupled to the illumination fiber(s) 62, e.g., via or within the handle 30, for delivering light through the illumination fiber(s) 62 and into the balloon 50.

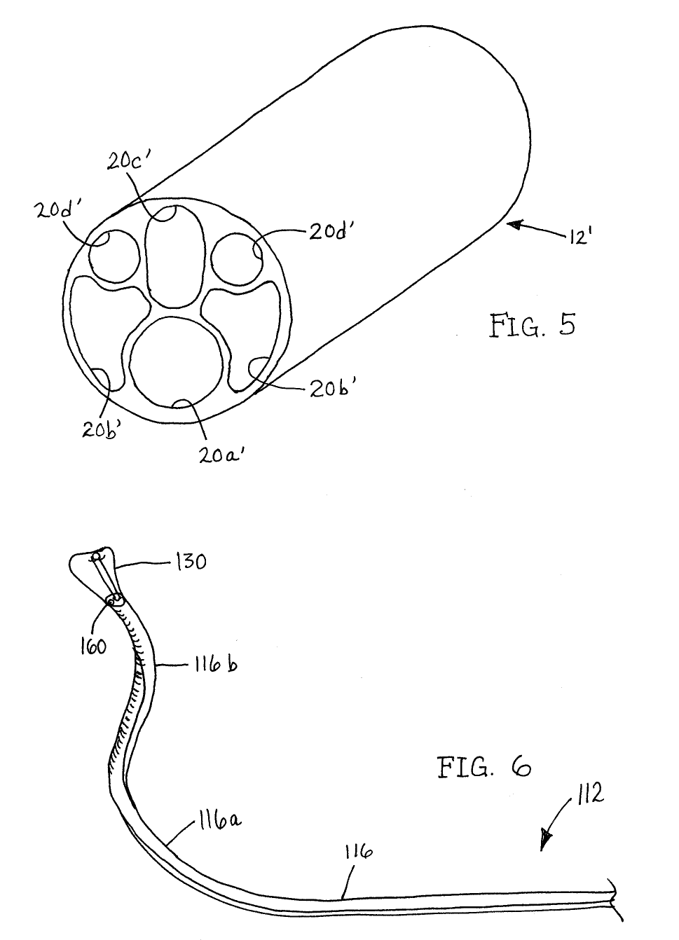

As shown in FIGS. 6-19, in various embodiments, the catheter 12 may include a steerable distal end 16 (and/or other portion) including a complex curved shape. This may be achieved by a combination of shape setting the distal end 16 and/or by providing a steering mechanism within the distal end 16. For example, the distal end 16 may include one or more bends, twists, or other deflections formed in the material of the distal end 16, e.g., to bias the distal end 16 towards the desired deflection(s). The bends, twists, or other deflections may extend through the entire steerable portion and/or to the distal tip of the catheter 12. Alternatively, the bends, twist, or other deflections may be located proximal to the distal tip, which may be substantially straight and/or may include a bend distally beyond the steerable portion. Thus, generally, the distal end 16 may assume a first, relaxed configuration, e.g., a substantially straight, curved, or other more complicated curvilinear shape when free from external forces, and may be directed to one or more additional configurations having complex curved, e.g., curvilinear, shapes.

Optionally, if the distal end 16 has a nonlinear shape in the first, related configuration, the catheter 12 may include a stiffening member (not shown), which may be advanced into the distal end 16 to at least partially straighten the distal end 16. For example, the stiffening member may be biased to a substantially straight (or other) shape and may have a rigidity greater than the distal end 16, while still being substantially flexible to accommodate bending. Thus, when the stiffening member is advanced into the distal end 16, the distal end 16 may become biased to assume the substantially straight (or other) shape of the stiffening member. When the stiffening member is withdrawn from the distal end 16 (e.g., using an actuator, not shown, in the handle 30), the distal end 16 may become biased to assume its nonlinear relaxed configuration.

In addition, the catheter 12 may be steerable, e.g., the distal end 16 may be controllably deflectable transversely relative to the longitudinal axis 18 using one or more pullwires or other steering elements. In the embodiment shown in FIGS. 4A-4E, the imaging fiber 64 may be used for steering the distal end 16 of the catheter 12 in one transverse plane (thereby providing one degree of freedom), as well as for obtaining images through the balloon 50. Alternatively, one or more separate pullwires (not shown) may be provided for steering the distal end 16 of the catheter 12, e.g., in two or more orthogonal planes (thereby providing two or more degrees of freedom).

With continued reference to FIGS. 4A-4E, the imaging fiber 64 (or other pullwire, not shown) may be attached or otherwise fixed relative to the catheter 12 at a location distal to or adjacent the distal end 16, and offset radially outwardly from a center of modulus of the catheter 12. If the construction of the catheter 12 is substantially uniform about the central axis 18, the center of modulus may correspond substantially to the central axis 18. If the construction of the catheter 12 is asymmetrical about the central axis 18, however, the center of modulus may be offset from the central axis 18 in a predetermined manner. As long as the optical fiber 64 (or other pullwire) is fixed at the distal end offset radially from the center of modulus, a bending moment may result when the imaging fiber 64 is pushed or pulled relative to the catheter 12 to steer the distal end 16.

For example, when the optical fiber 64 is pulled proximally or pushed distally relative to the catheter 12, e.g., from the proximal end 14 of the catheter 12, a bending force may be applied to the distal end 16, causing the distal end 16 to curve or bend transversely relative to the central axis 18. Optionally, the degree of steerability of the distal end 16 may be adjustable, e.g., to increase or decrease a radius of curvature of the distal end 16 when the imaging fiber 64 is subjected to a predetermined proximal or distal force. In addition or alternatively, one or more regions of the catheter 12 may be set to be steerable in a predetermined manner.

For example, any of the catheters 12 described herein may include a steering adjustment member, which may be an elongate member 80 slidably disposed within the catheter 12. For example, as shown in FIG. 4A, the elongate member 80 may be slidable along lumen 20c for selectively directing a portion of the imaging fiber 64 between different regions of the lumen 20c, e.g., to change a bending moment and/or bendable length of the distal end 16. Similar to the imaging fiber 64 or other pullwire (not shown), the steering adjustment member 80 may be sufficiently flexible not to substantially interfere with bending of the distal end 16 and may have sufficient tensile and/or column strength to allow the steering adjustment member 80 to be pushed and pulled within the catheter 12, e.g., from the proximal end 14. Additional information and embodiments of a steering adjustment member may be found in application Ser. No. 11/062,074, incorporated by reference above.

Returning to FIG. 1, the handle 30 may include one or more steering controls (not shown) for controlling the ability to steer the distal end 16 of the catheter 12. For example, the handle 30 may include an actuator 32 that may be coupled to the optical fiber 64 such that proximal movement of the actuator applies a proximal force to the optical fiber 64. The resulting bending moment causes the distal end 16 of the catheter 12 to bend into a curved shape. Optionally, the actuator may be biased, e.g., to return the distal end 16 of the catheter 12 to a generally straight configuration when the actuator is released. Alternatively, the actuator may include a resistive mechanism (not shown), which may allow the distal end 16 to maintain a curved configuration once the actuator is moved to a desired position to steer the distal end 16. Optionally, other actuator(s) may be provided for controlling one or more other pullwires and/or steering adjustment members.

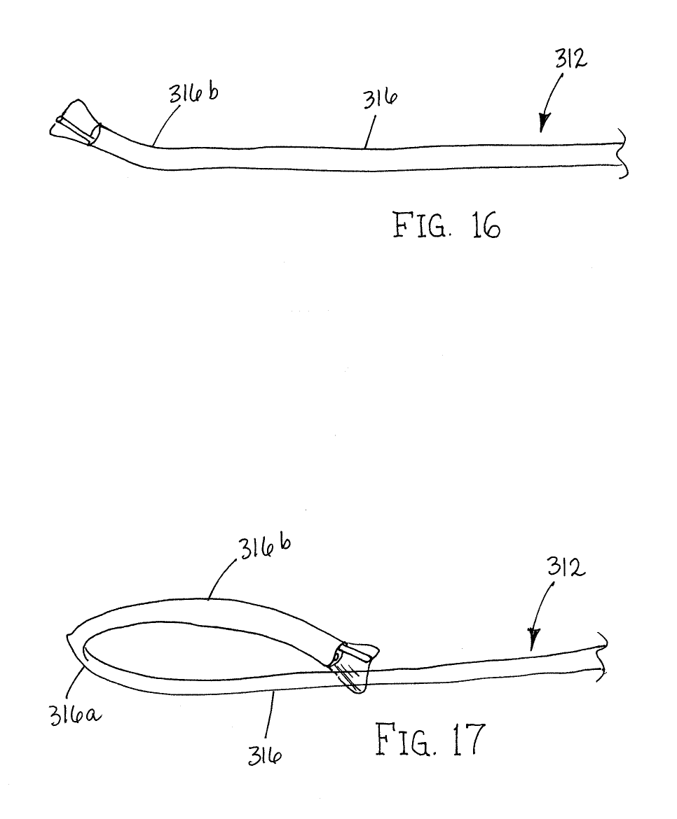

Turning to FIGS. 6-17, various embodiments of distal ends of catheters are shown that may be directed from relaxed configurations to one or more complex curved configurations. Generally, the catheter may include a proximal end (not shown), which may be substantially rigid and/or may otherwise remain substantially straight relative to the distal end. The distal end may include a preset shape, e.g., a substantially straight shape, a simple bend, or a more complicated curvilinear shape set in the material. For example, as shown in FIGS. 15-19, in one embodiment, in the relaxed configuration, the distal end 316 may include a bend of approximately thirty degrees (30°) relative to a central axis defined by the portion of the catheter 312 proximal to the bend, and approximately ninety degrees (90°) out of plane as compared to a plane defined by the portion proximal to the bend.

Turning to FIGS. 6-9, a first embodiment of a distal end 116 of a catheter 112 is shown. In this embodiment, the material of the distal end 1 16 may be twisted about a central axis 118 and the twist set into the material, e.g., by heat treating or other process. In addition, the catheter 112 includes a pullwire (not shown) that may extend through the distal end 116 offset from the central axis 118. Optionally, the catheter 112 may include an internal steering adjustment member (not shown), similar to the member 80 shown in FIG. 4B and described above.

As shown in FIG. 9, the distal end 116 may be biased to assume a substantially straight shape in the relaxed configuration. However, as shown in FIGS. 6-8, when the pullwire is actuated, the distal end 116 may experience a bending moment, causing the distal end 116 to bend into a complex curved shape. In particular, as best seen in FIG. 6, the distal end 116 may include a first bending portion 116a, which may be a simple arc created by the bending moment, and a second spiral portion 116b caused by the twist in the distal end 116.

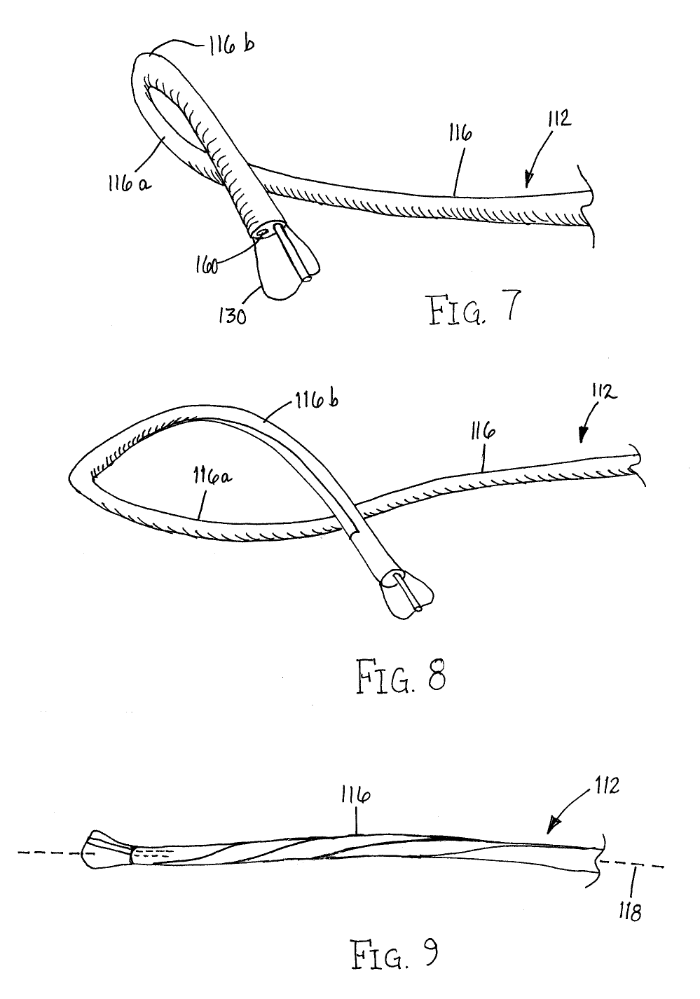

With continued reference to FIG. 6, the first bending portion 116a may bend clockwise within a plane of the page, while the second spiral portion 116b extends upwardly out of the plane. This configuration of complex curved shape is described elsewhere herein as the “left hand rule.” Turning to FIG. 7, which shows the configuration of FIG. 6 along the angle of the first bending portion 116a, the orientation of the balloon 130 and imaging assembly 160 extends across this plane. FIG. 8 shows the configuration of FIGS. 6 and 7 from an end of the catheter 112. The configuration shown in FIGS. 6-8 may facilitate directing the balloon 130 towards a coronary sinus from a superior approach into the right atrium, as described elsewhere herein.

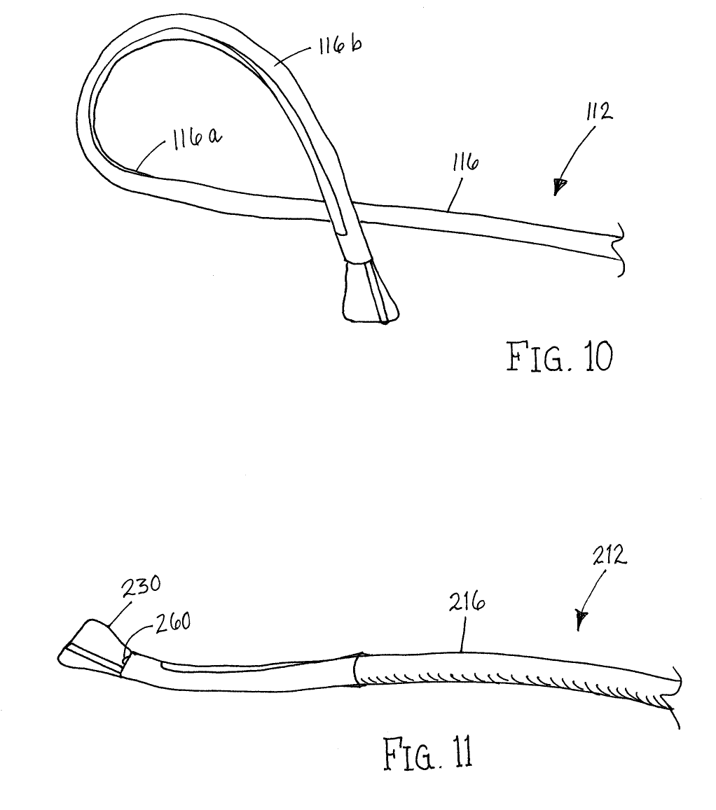

Turning to FIG. 10, another configuration of the distal end 116 is shown. In this configuration, an internal steering adjustment member (not shown) has been withdrawn from the distal end 116, thereby causing the arc defined by the distal end 116 to define a relatively larger radius than that shown in FIGS. 6-8. Thus, by adjusting the steering adjustment member, a variety of complex curved configurations may be attained, which may facilitate introducing the distal end 116 into a target body lumen, e.g., as described with reference to FIGS. 20-23 below.

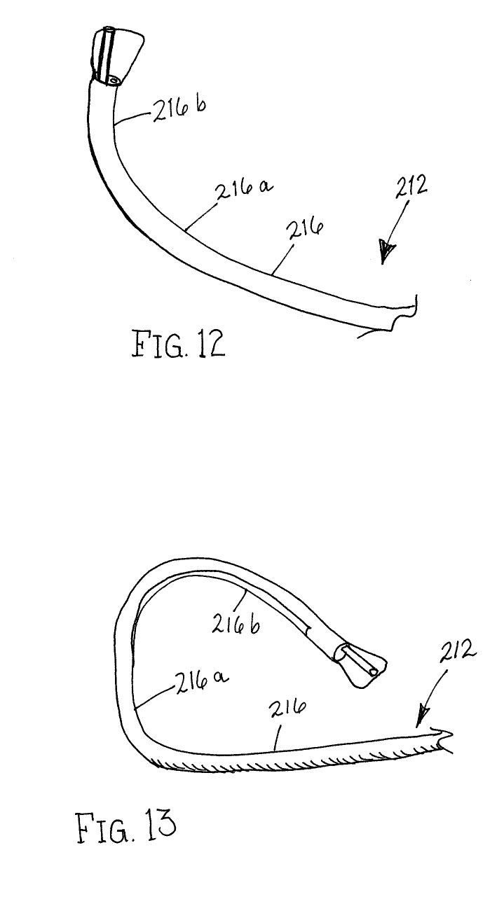

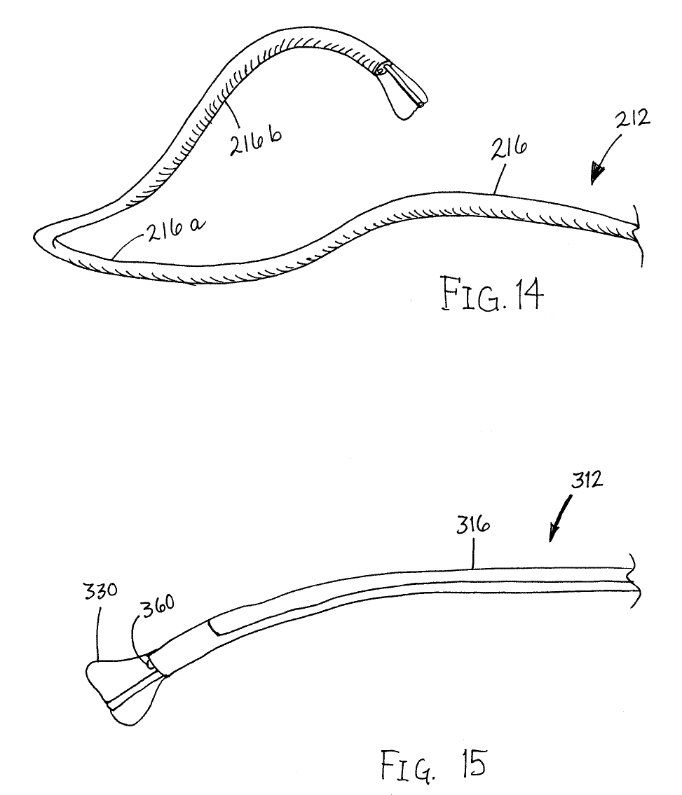

Turning to FIGS. 11-14, a second embodiment of a distal end 216 of a catheter 212 is shown, which may be constructed generally similar to the previous embodiments described herein. FIG. 11 shows the distal end 216 in a substantially straight, relaxed configuration. In FIGS. 12 and 13, the distal end 216 has assumed a complex curved configuration, e.g., upon actuation of a steering mechanism (not shown). The complex curved configuration may include a gradual curved portion 216a that extends out of a plane, and a twisted portion 216b distal to the gradual curved portion 216a. As best seen in FIG. 13, the twisted portion 216b may resemble a portion of a corkscrew or helical shape, while the gradual curved portion 216a may have a generally “J” shape. Thus, the distal end 216 may resemble a corkscrew or helical twist in the complex curved configuration that may allow the catheter 212 to extend through varying geometrical shapes of anatomy, e.g., through a patient's right atrium into a coronary sinus. Optionally, the gradual curved portion 216a and the twisted portion 216b may substantially overlap, or the twisted portion 216b may extend through substantially all of the deflectable region of the catheter and still achieve a desirable shape.

Turning to FIG. 14, another complex curved configuration is shown from a top-down view, showing the distal end 216 with a complex shape combining an elongated gradual curved portion 216a and a helical, twisted portion 216b. The ratio and distance of the gradual curved portion 216a combined with the twisted portion 2116b is shown in exemplary configurations, but may encompass various other combinations.

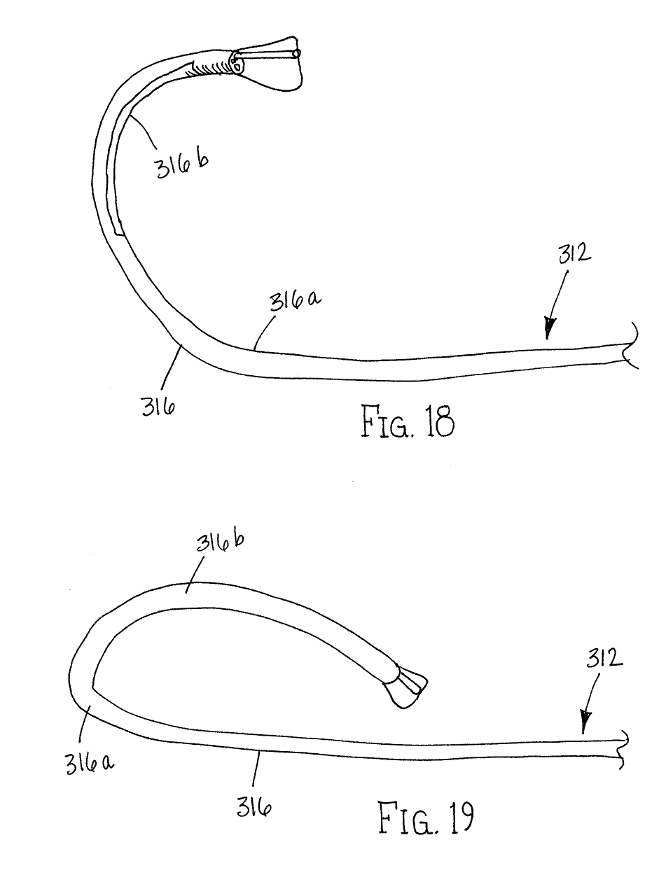

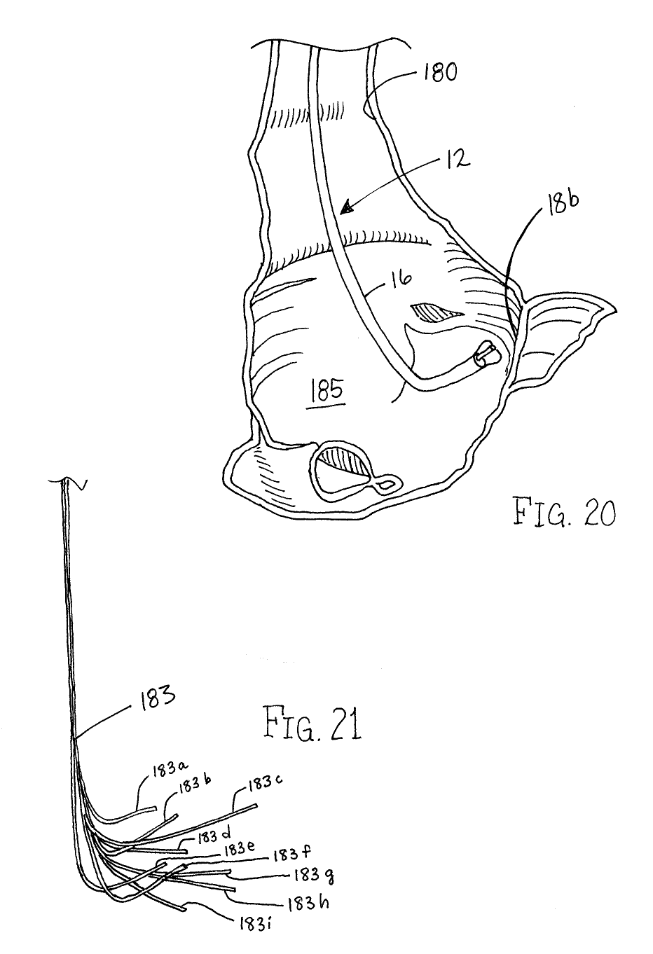

Referring to FIG. 15-19, a third embodiment is shown of a catheter 212 that generally includes a proximal end (not shown) and distal end 316 sized for introduction into a body lumen, similar to the previous embodiments. The distal end 316 is shown in a relaxed configuration in FIGS. 15 and 16, and in a complex curved configuration in FIGS. 17 and 18. In the relaxed configuration, the distal end 316 includes a slightly curved shape in two planes, as shown in FIGS. 15 and 16. In the complex curved configuration, the distal end 316 assumes a curvilinear shape, including a first gradual curve 316a within a first plane, and a second smaller curve 316b within a single plane that intersects the first plane. FIG. 19 shows the distal end 316 in another complex curved configuration created by actuating an internal steering adjustment member, as described elsewhere herein.

Optionally, any of the embodiments described herein may include a substantially transparent expandable member on a distal end of the catheter or other apparatus, an optical imaging assembly on the distal end, and/or other features or structures, depending upon the intended use for the apparatus, e.g., as described above. The preformed distal end may allow an optical imaging assembly to keep its field of view, and any image therein, in alignment with the distal end of the apparatus.

Turning to FIG. 20, a cross-sectional view of a heart is shown, including a right atrium 185, superior vena cava 180, and coronary sinus 186. This exemplary view is merely illustrative, and may not reflect any particular patient's anatomy. As discussed elsewhere herein, patients' geometries and trajectories, e.g., from the superior vena cava through the right atrium and in to the coronary sinus, may vary widely. One of the various complex curved configurations of a catheter 12 is shown that may be capable of being directed through the right atrium 185 into the coronary sinus 186.

Generally, the catheter 12 may be advanced from an entry site, e.g., a percutaneous puncture in a peripheral vein or other vessel, into the superior vena cava 180. From the superior vena cava 180, the catheter 12 may enter the right atrium 185, which may diverge anatomically relative to the coronary sinus 186. For each patient, the connecting region of the right atrium 185 between the superior vena cava 180 and the coronary sinus 186 may define a pathway, which may be modeled in order to define connect the superior vena cava pathway 180 with the coronary sinus trajectory pathway 183, as measured from the individual patient being treated.

Referring to FIGS. 19-21, several lines 183 are shown, which may represent different pathways necessary to pass from the superior vena cava, through the right atrium and into the coronary sinus for a particular patient. As mentioned previously, the patients' geometries and trajectories, from the superior vena cava through the right atrium and into the coronary sinus, may vary widely. Nine exemplary coronary sinus trajectory pathways 183a-183i are represented in FIG. 19, as modeled from actual patient data. FIG. 20 shows the pathways 183 from a top-down view, where the superior vena cava is projected in a plane extending substantially perpendicularly out of the page and the juncture to the coronary sinus is visible. FIG. 21 is another side perspective view showing the nine exemplary trajectory pathways 183.

Thus, as can be seen, the necessary pathway to navigate successfully from the superior vena cava to the coronary sinus may vary dramatically, which can make navigation through a particular patient's heart difficult to predict, and, in practice, difficult to accomplish. The complex curved configurations achievable with the apparatus described herein may facilitate such navigation, but providing a shape configured to place the tip of the apparatus within close proximity to the coronary sinus. By then steering or otherwise manipulating the apparatus, the coronary sinus may be located and/or accessed more easily.

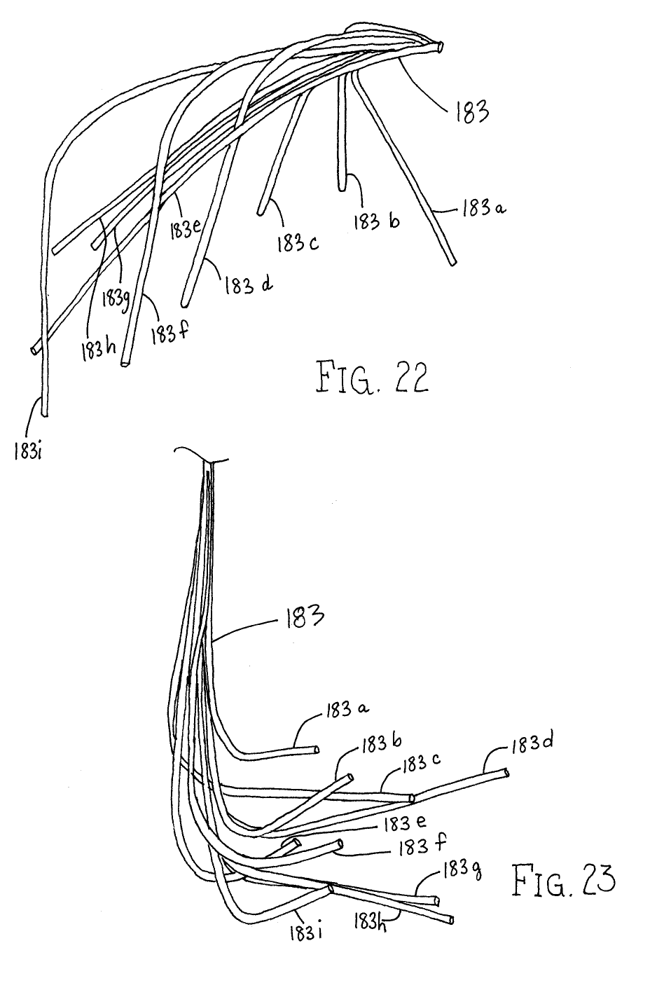

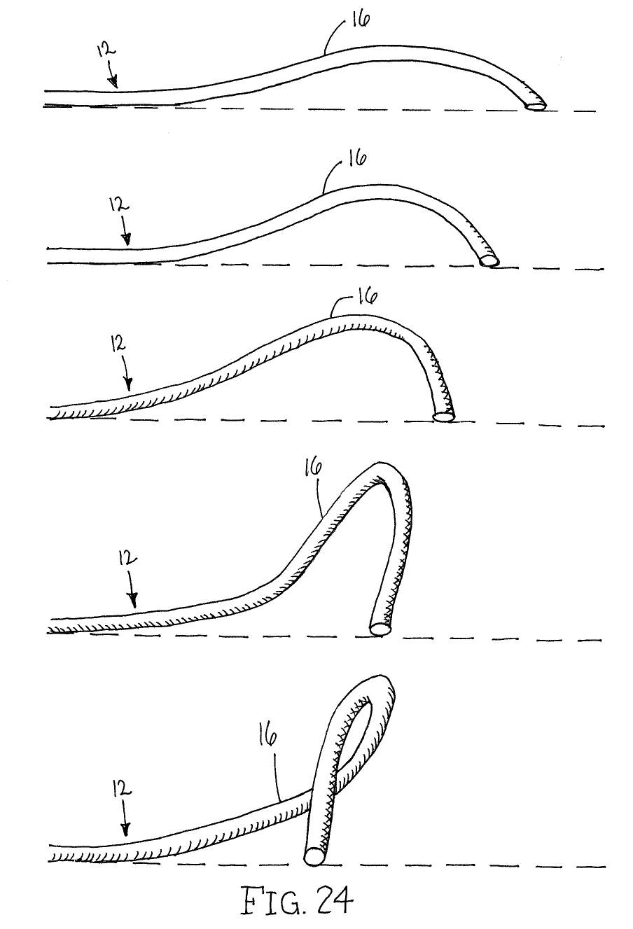

FIG. 22 shows a series of configurations that another embodiment of a distal end 16 of a catheter 12 may assume as an actuator is used to control a steering mechanism (not shown) of the catheter 12. In this embodiment, the distal end 16 of the catheter 12 may include a twisted portion, which may define a helical shape in the final, fully actuated configuration. In the embodiment shown, the catheter 12 may be constructed with a twist, preferably linear in pitch, such that deflection of a distal tip in at least one plane is substantially linear throughout the majority of deflection. Alternatively speaking, the steerable distal end 16 of the catheter 12 may deflect substantially orthogonally relative to the direction of deflection in at least one plane such that the tip position defines a substantially linear pathway throughout the majority of deflection. This configuration may provide at least two benefits. First, the catheter tip may be maintained within the open space of the atrium as it transitions from a straight, relaxed configuration to a final desired complex curved configuration. This may facilitate advancing or otherwise manipulating the distal end 16 through the right atrium without substantial interference from structures or other features of the wall of the right atrium or body cavity. Second, the substantially linear tip deflection in at least one plane may facilitate the user understanding the position of the distal end 16 of the catheter as it assumes the complex curved configuration, e.g., while monitoring the catheter 12 using a two dimensional reference system, such as fluoroscopy or other external x-ray imaging systems.

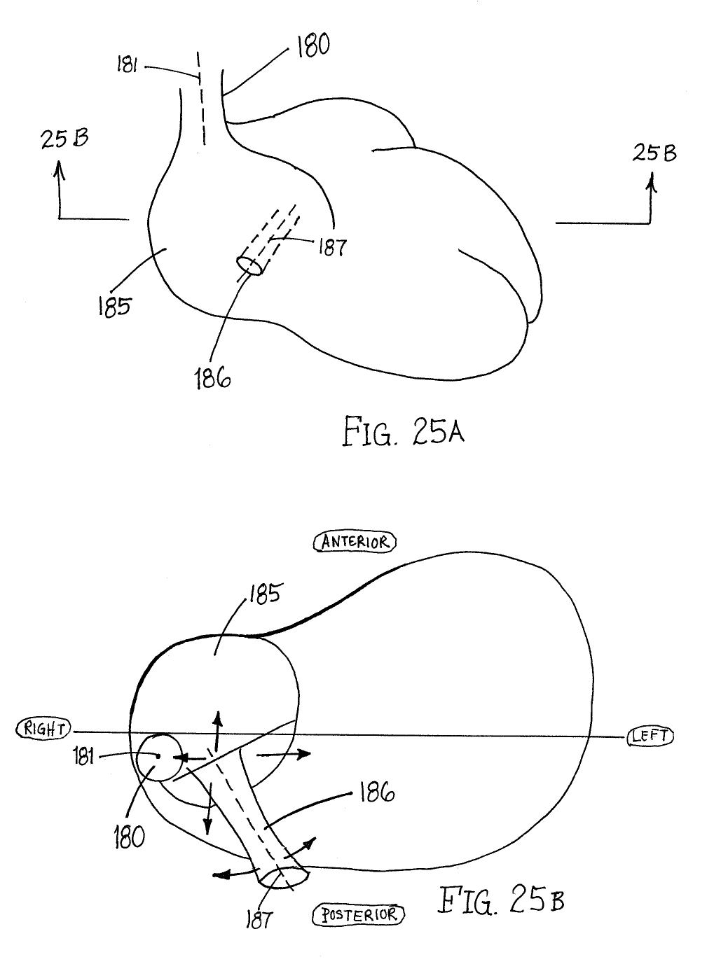

Turning to FIGS. 25A and 25B, a heart is shown that includes a superior vena cava 180 defining a longitudinal axis 181, a right atrium 185, and a coronary sinus 186 defining a longitudinal axis 187. As can be best seen in FIG. 25B, the axes 181, 187 of the superior vena cava 180 and coronary sinus 186 are generally offset from one another. Thus, when a simple curved catheter (not shown) is extended along the axis 181 of the superior vena cava 180 into the right atrium 185, the vector along which the tip of the catheter travels may avoid the coronary sinus entirely.

If, in some particular anatomy, the coronary sinus 186 is located towards the right side of the patient (towards the superior vena cava 180) or more posteriorly, the axes 181, 187 may become closer to intersecting, and a simple curved catheter may have a better chance of approximating the coronary sinus 186. If the coronary sinus 186 is located to the patient's left or more anteriorly, the axes 181, 187 may diverge even further, making approximating the coronary sinus 186 more difficult. Similar difficulties may emerge if the coronary sinus is oriented more clockwise or if the axis 187 of the coronary sinus 186 does not lie within the plane of the cross-section of FIG. 25B (e.g., as can be seen in FIG. 25A). These drawings demonstrate the difficulties in navigating through the right atrium 185 from the superior vena cava 180 into the coronary sinus 186, a problem that the apparatus and methods described herein may be particularly suited to overcome.

While the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents and alternatives falling within the scope and spirit of the description and limited only by the claims.

We claim:

1. An apparatus for accessing a body lumen within a patient's body, comprising:
a tubular member comprising a proximal end, a distal end sized for introduction into a body lumen, and a distal portion comprising material formed to bias the distal portion to assume a first relaxed configuration;
a steering element extending from the proximal end to the distal portion; and
an actuator for applying an axial force to the steering element, thereby causing the distal portion to assume a complex curved configuration.

2. The apparatus of claim 1, wherein the complex curved configuration comprises a curvilinear shape.

3. The apparatus of claim 2, wherein the curvilinear shape extends along at least first and second planes that intersect one another.

4. The apparatus of claim 1, wherein, in the complex curved configuration, the distal portion comprises a first curved portion comprising an arc defining a first plane and radius of curvature, and a second curved portion comprising a curved shape extending out of the first plane.

5. The apparatus of claim 4, wherein the second curved portion comprises a spiral shape extending out of the first plane.

6. The apparatus of claim 5, wherein the spiral shape comprises a left hand helix.

7. The apparatus of claim 5, wherein the spiral shape comprises a right hand helix.

8. The apparatus of claim 4, wherein the first curved portion curves clockwise along the first plane, and wherein the second curved portion extends upwardly out of the first plane.

9. The apparatus of claim 4, further comprising a steering adjustment member movable within the distal portion for changing at least one of the radius of curvature of the first curved portion and the curved shape of the second curved portion.

10. The apparatus of claim 4, wherein the first curved portion is located proximal to the second curved portion.

11. The apparatus of claim 4, wherein the second curved portion is coextensive with the first curved portion.

12. The apparatus of claim 1, wherein the distal portion comprises a first twisted portion, and wherein the steering element extends along the first twisted portion helically around a longitudinal axis of the tubular member, whereby the first twisted portion is biased to assume a helical shape in the complex curved configuration.

13. The apparatus of claim 1, wherein the distal portion is configured such that a distal tip of the distal end travels along a linear path as the distal portion is directed from the relaxed configuration to the complex curved configuration.

14. The apparatus of claim 1, further comprising a substantially transparent expandable member carried by the distal end of the tubular member, the expandable member being expandable from a collapsed condition to an expanded condition; and
an optical imaging assembly carried by the distal end of the tubular member and at least partially surrounded by the expandable member, the optical imaging assembly for imaging the tissue structure beyond the distal end through the expandable member.

15. The apparatus of claim 1, wherein the distal end comprises a distal tip extending distally beyond the distal portion.

16. The apparatus of claim 11, wherein the distal tip comprises a bend.

17. The apparatus of claim 11, wherein the distal tip is substantially straight.

18. The apparatus of claim 11, wherein the distal tip comprises a tubular extension.

19. An apparatus for accessing a body lumen within a patient's body, comprising:
a tubular member comprising a proximal end, a distal end sized for introduction into a body lumen, the distal end comprising flexible material formed to bias the distal end to assume a first substantially straight configuration;
a steering element extending from the proximal end to the distal end; and
an actuator for applying an axial force to the steering element, thereby causing the distal end to assume a complex shaped configuration, the distal end comprising a first curved portion comprising an arc defining a first plane and radius of curvature, and a second portion distal to the first curved portion and extending out of the first plane in the complex shaped configuration.

20. The apparatus of claim 19, wherein the second portion comprises a curved shape.

21. The apparatus of claim 19, wherein the second portion comprises a helical shape.

22. The apparatus of claim 21, wherein the helical shape comprises a left hand helix.

23. The apparatus of claim 19, wherein the arc of the first curved portion extends clockwise along the first plane and the second portion extends upwardly out of the first plane, thereby approximating a pathway extending from a superior vena cava to a coronary sinus within a heart.

24. The apparatus of claim 19, further comprising a steering adjustment member extending between the proximal and distal ends, and an actuator coupled to the steering adjustment member for changing at least one of the radius of curvature of the first curved portion and an angle between the second portion and the first plane when the steering adjustment member is operated using the actuator.

25. A method for making a steerable portion of a tubular member sized for introduction into a body lumen of a patient, comprising:
providing an elongate tubular body comprising a first end and a second end sized for introduction into a body lumen;
setting a predetermined shape in the tubular body comprising at least one of a twist about a longitudinal axis of the tubular body and a curve;
disposing a steering mechanism through the tubular body such that; and
fixing one end of the steering mechanism adjacent the first end of the tubular body, such that an axial force applied to the other end of the steering mechanism causes the tubular body to move from a relaxed configuration to a complex shaped configuration.

26. The method of claim 25, wherein the tubular body comprises a tubular extrusion and an outer layer surrounding the extrusion.

27. The method of claim 25, wherein the tubular body comprises a tubular coextrusion and an outer layer surrounding the coextrusion.

28. The method of claim 25, wherein the tubular body comprises a core including first and second side portions having different durometers and an outer layer surrounding the first and second side portions.

29. The method of claim 25, further comprising disposing a steering adjustment member within the tubular body, steering adjustment member being movable for modifying a bending characteristic of the tubular body.

30. An apparatus for accessing a body lumen within a patient's body, comprising:
a tubular member comprising a proximal end, a distal end sized for introduction into a body lumen, a distal portion capable of assuming a first relaxed configuration and a second complex shaped configuration, and a distal tip beyond the distal portion;
a steering element extending from the proximal end to the distal portion; and
an actuator for applying an axial force to the steering element, thereby causing the distal portion to move between the relaxed and complex shaped configurations, the distal portion configured such that the distal tip travels along a substantially straight path as the distal portion moves between the relaxed and complex shaped configurations.

31. A method for accessing a coronary sinus of a heart including a superior vena cava and a right atrium, comprising:
directing a distal portion of a tubular member into the right atrium via the superior vena cava;
directing the distal portion to a complex shaped configuration within the right atrium, thereby disposing a distal tip of the tubular member in proximity to the coronary sinus. manipulating the tubular member to direct the distal tip towards the coronary sinus.

32. The method of claim 31, wherein the distal tip travels along a substantially straight line as the distal portion is directed to the complex shaped configuration.

33. The method of claim 31, further comprising expanding an expandable member on the distal tip and directing the expandable member into contact with a wall of the right atrium.

34. The method of claim 33, wherein manipulating the tubular member comprises directing the expandable member along the wall of the right atrium and imaging the wall through the expandable member to identify the coronary sinus.

35. The method of claim 31, wherein the distal portion comprises a first curved portion defining a plane and a second portion extending out of the plane in the complex shaped configuration, thereby approximating a curvilinear pathway from the superior vena cava towards the coronary sinus.
